(19) United States
(12) Patent Application Publication
(10) Pub. No.: US 2017/0202454 A1
(43) Pub. Date: Jul. 20, 2017

(54) Eye Examining System And Method For Eye Examination

(71) Applicant: Oculus Optikgeraete GmbH, Wetzlar (DE)

(72) Inventors: Andreas Steinmueller, Wettenberg (DE); Stephan Degle, Jena (DE)

(21) Appl. No.: 15/405,922

(22) Filed: Jan. 13, 2017

(30) Foreign Application Priority Data
Jan. 14, 2016 (DE) 10 2016 000 232.8

(51) Int. Cl. A61B 3/103; A61B 3/00; A61B 3/032; A61B 3/14

(52) U.S. Cl. CPC A61B 3/103; A61B 3/14; A61B 3/0025; A61B 3/0041; A61B 3/032; A61B 3/0091

(57) ABSTRACT

The invention relates to an eye examining system and a method for examining a subject's eyes by means of an eye examining system, eye examination symbols being made visible to at least one of the subject's eyes using a display device of the eye examining system, the display device comprising a camera device having a camera by means of which the subject's eyes can be recorded, the display device comprising an illumination device having a light source by means of which the subject's eyes can be illuminated, a light distribution being able to be recorded in the pupil of the subject's eye by means of the camera device of the display device, the eye examining system comprising a control apparatus by means of which an objective refraction of the eye can be determined via the light distribution in the pupil.

### CROSS REFERENCE TO RELATED APPLICATION

This application claims the priority benefit of German Patent Application No. 10 2016 000 232.8 filed on Jan. 14, 2016, which is fully incorporated herein by reference.

### BACKGROUND OF THE INVENTION

The invention relates to an eye examining system and a method for examining a subject's eyes, eye examination symbols being made visible to at least one of the subject's eyes using a display device of the eye examining system, the display device comprising a camera device having a camera by means of which the subject's eyes can be recorded, the display device comprising an illumination device having a light source by means of which the subject's eyes can be illuminated.

Such eye examining systems are well known and are regularly used for conducting eye examinations. The known display devices or rather monitors comprise a control device by means of which a user can control displaying eye examination symbols on the monitor. Depending on the type of monitor, the monitor can also comprise a linear or circular polarization. The polarization of the monitor is regularly used for conducting eye examinations in conjunction with a trial frame or a phoropter. The camera device can be used for measuring the eyes, for example, although measuring the eyes is barely possible when simulating mesopic or scotopic vision conditions due to the illumination conditions.

A disadvantage of the known eye examining systems for determining a subjective refraction by means of eye examination symbols is that a user has to initially take tentative guesses at or iteratively approach the subject's refraction via displaying various different eye examination symbols in conjunction with reference lenses of a phoropter or a trial frame. This process regularly takes up quite a lot of time. A subject's refraction can be objectively determined by means of an aberrometer or an autorefractometer, however, the refraction subjectively perceived by the subject as being ideal may differ to the objectively determined refraction which is why subjectively determining the refraction is indispensable. Moreover, an aberrometer is not always available and comparatively expensive so that the refraction can be subjectively determined without having previously conducted a measurement with the aberrometer.

### SUMMARY OF THE INVENTION

The object of the invention at hand, therefore, is to propose an eye examining system and a method for examining a subject's eyes with an eye examining system by means of which eye examinations can be conducted quicker.

The eye examination device according to the invention for examining a subject's eyes comprises a display device by means of which eye examination symbols are made visible to at least one of the subject's eyes, the display device comprising a camera device having a camera by means of which the subject's eyes can be recorded, the display device comprising an illumination device having a light source by means of which the subject's eyes can be illuminated, a light distribution being able to be recorded in the pupil of the subject's eye by means of the camera device of the display device, the eye examining system comprising a control apparatus by means of which an objective refraction of the eye can be determined via the light distribution in the pupil.

A fundus or rather a retina of the subject's eye is illuminated by means of the light source. A blurred image on the eye's retina is thereby the result of an ametropic eye. This image is recorded by the camera of the camera device through the pupil. This recorded light distribution in the pupil or rather the pupil reflection are each different in dependence of the existing ametropia of the corresponding eye.

The control apparatus or the control device, respectively, is preferably a computer which processes the light distribution in the pupil recorded by the camera by means of image processing. The control apparatus can thus determine the light distribution in the pupil and can, for example, compare the light distribution to a normal light distribution of a healthy eye or a comparison group of subjects, respectively. Optionally, the control apparatus can also compare the light distribution in the pupil of the subject's eye to light distributions of myopic eyes stored in a database by simply comparing images. Thus, the control apparatus can determine or evaluate, respectively, an objective refraction of the eye which is not as exact as a measurement taken with an aberrometer but suffices for conducting a subjective eye examination with eye examination symbols adjusted accordingly in size. An iterative approach, which would otherwise be necessary, to the subjective refraction by displaying various different eye examination symbols is therefore considerably reduced. Furthermore, an objective refraction and a subjective refraction can be determined by means of just the one eye examining system, whereby a separate measurement using an aberrometer is no longer necessary. The eye examining system can therefore be operated at little cost.

The display device, the camera and the light source can be arranged in a shared housing of the eye examining system. The eye examining system can then be handled particularly easily since there is no need for multiple devices which are coupled but distanced to each other for conducting an eye examination.

The display device can be a stationary display device for testing farsightedness, whose display surface size is designed for eye examinations conducted at a seeing distance from 3 m to 10 m, preferably 4 m to 8 m, and/or a display device for testing nearsightedness for mobile use, whose display surface is designed larger for eye examinations conducted at a seeing distance from 10 cm to 3 m, preferably from 30 cm to 1 m. The display device for testing farsightedness can then be used for displaying eye examination symbols for testing farsightedness and the display device for testing nearsightedness can be used for displaying eye examination symbols for testing nearsightedness. Both display devices can be used separately or in conjunction with each another as an eye examining system. The display device for testing farsightedness can preferably be set up in a stationary manner or be mounted to a wall relative to the subject at the seeing distance stated above. If the subject is positioned at a defined seeing distance relative to the display device for testing farsightedness when conducting an eye examination, the seeing distance to the display device for testing farsightedness can be exactly determined. Correspondingly, a display surface size of the display device for testing farsightedness can then be many times larger than a display surface size of the display device for testing nearsightedness, since the eye examination symbols displayed on the display surface of the display device for testing farsightedness may possibly be comparatively larger. The display device for testing nearsightedness can also be used movably so that it can be placed or held relative to the subject's eyes at any possible distance within the seeing distance stated above by a user or the subject. The display device for testing farsightedness as well as the display device for testing nearsightedness can be remote controlled by a user via the control apparatus. The control apparatus can comprise a control device for remote controlling.

The display device can comprise a backlit monitor, the display device being preferably designed as a type of television, monitor or tablet computer. A control device of the control apparatus can communicate wirelessly with the display device by means of a Wi-Fi or Bluetooth connection. The control device, however, can also be a permanently installed computer or laptop on which software for controlling the display device can be executed. The backlit monitor can be an LCD monitor whose monitor luminance can preferably be proportionately adjusted to a surrounding luminance. The monitor can also be designed having linear or circular polarization, for example, or a different installation which can be used for image separation.

Furthermore, it can be provided that the monitor forms a light source. The monitor can then be partially or entirely operated with such a high monitor luminance that the monitor alone illuminates the subject's eyes.

The light source can be an infrared light source. The subject's eyes can also be illuminated by means of the infrared light source, in particular in the cases when eye examinations are conducted under isotopic or scotopic lighting conditions. Due to a reduced surrounding brightness, it is therefore difficult to record a subject's eyes with a camera device when conducting certain eye examinations. By means of the infrared light source, the subject's eyes can be recorded independently of a surrounding brightness having infrared lighting by means of a correspondingly adjusted camera device or rather an infrared camera. Advantageously, blinding the subject can also be avoided by using infrared light to illuminate the eyes. In total, it is thus possible to discover whether the objective refraction determined under mesopic vision or scotopic vision conditions varies from a refraction subjectively determined under the same lighting conditions and consequently equal pupil diameters.

Furthermore, it can be provided to movably realize the camera device and/or the infrared light source in a storage position in the display device or in a receiving position outside of the display device. The camera device and/or the infrared light source can be arranged at the display device or the monitor, respectively, so that the camera can be either lowered to take up a storage position, for example behind the monitor, or be moved to take up a receiving position beside the monitor for receiving a camera as required. Moving the camera from the storage position to the receiving position and vice versa can occur via a driving unit of the camera device. If a comparatively large camera is being used, a deflection prism can be provided so that the camera can be arranged behind the monitor so as to take up little space.

Moreover, the eye examining system can comprise a phoropter or a trial frame. This makes it possible to determine a subjective refraction of a subject's eyes, respectively. The phoropter and the trial frame can also comprise color filters or polarization filters, which are each adjusted to a color display and/or a polarization of the monitor, so that monocular and binocular eye examinations can be conducted. If, for example, a phoropter or a trial frame is provided having linear or circular polarization, the display device of the eye examining system can be chosen so as to correspond to the polarization of the phoropter or the trial frame. Correcting a polarization, for example by means of a λ/4 filter, is therefore not required.

The light source can be formed from a light-emitting diode which can be eccentrically arranged relative to a lens of the camera or its optical axis, respectively. Only by eccentrically arranging the light-emitting diodes relative to the optical axis of the camera is it possible to illuminate the eye at an angle different to the optical axis of the camera. If the optical axis of the camera perfectly aligns with the visual axis of the eye, an image can be detected on the retina through the pupil with a camera. The objective refraction can therefore be determined in a particularly easy manner based on the so-called photo refraction principle.

A plurality of light-emitting diodes can form the light source and coaxially surround the lens of the camera. It can also be provided that the light source can comprise several infrared (IR) light-emitting diodes. The light sources can be arranged directly adjacent to the camera. Four light-emitting diodes, for example, can be equidistantly arranged beside the camera or the infrared camera, respectively, relative to the camera. It is generally possible to also arrange light-emitting diodes in a frame of the display device.

In the method according to the invention for examining a subject's eyes with an eye examining system, eye examination symbols are made visible or rather shown to at least one of the subject's eyes using a display device of the eye examining system, the subject's eyes being recorded by means of a camera of a camera device, the subject's eyes being illuminated by means of a light source of an illumination device of the display device, a light distribution being recorded in the pupil of the subject's eye by means of the camera device of the display device, an objective refraction of the eye being determined from the light distribution in the pupil by means of a control apparatus of the eye examining system. The advantageous description of the eye examining system according to the invention is referred to for the advantages of the method according to the invention.

The display device, the light source and/or the camera can be controlled by means of the control apparatus. The control apparatus can further comprise a control device which can be used for remote controlling the display device, the light source and/or the camera. Determining or evaluating, respectively, the objective refraction of the eye by means of the control apparatus can be carried out in the display device or the control device by means of a computer or an installation for processing data, respectively. If the control apparatus controls the display device, the light source and the camera, a mostly automated eye examination can be conducted by the control apparatus.

In a first step, an objective refraction of the subject's eye can be determined by means of the eye examining system; in a second step, eye examination symbols can be made visible to the subject's eye for determining a subjective refraction by means of the eye examining system, the second step being able to directly follow the first step. Before determining a subjective refraction by showing eye examination symbols, an objective refraction can be determined. This measurement can be conducted essentially automated and without the support of a user.

The eye examination symbols shown to the subject can therefore be adjusted to the objectively measured refraction. The eye examination symbols can be made visible in a size which is adjusted to the objectively measured refraction of the subject's eye. Determining the subjective refraction therefore is carried out solely for verifying the objectively measured refraction. This verification can also be carried out when considering a pupil diameter. Since the objectively measured refraction was determined having a large pupil diameter at consistent lighting conditions, for example, it can be assumed when subjectively verifying the refraction that the pupil diameter is essentially the same due to an unchanged surrounding luminance. Provided the surrounding luminance or a monitor luminance, as well, changes between measuring the objective refraction and measuring the subjective refraction, the pupil diameter can also comparatively change so that possibly a subjectively measured refraction is yielded which differs from the objectively measured refraction. It can thus be provided that a pupil diameter or a surrounding luminance, respectively, and/or a monitor luminance are each taken into consideration when determining the refraction.

Therefore, the eye examination symbols for subjectively determining refraction can be chosen by the control apparatus in dependence of the objective refraction. In particular, the eye examination symbols can be shown in a size adjusted to the measuring distance if a seeing distance or a measuring distance, respectively, is known. The eye examination symbols adjusted in size can be shown automatically or manually via the control apparatus or via a user, respectively. Thus, the eye examination symbols are always shown in the actually required size and the time required for conducting an eye examination is considerably reduced.

In particular, determining the objective refraction can be carried out by means of the photo refraction principle.

The eye can also be illuminated with light-emitting diodes of the illumination device simultaneously or sequentially, a corresponding pupil reflection being able to be recorded by the camera. If the light-emitting diodes are arranged at a relative distance to one another and are arranged coaxially to a lens of the camera, it becomes possible to illuminate the eye's retina from different illumination positions so that a sequence of different pupil reflections can be recorded by the camera. This corresponding light distribution in the pupil can then be used for determining a relatively exact objective refraction of the eye.

For determining the objective refraction of a subject's eye, a fixation stimulus can be shown on a monitor of the display device or at a different position of the eye examining system. The fixation stimulus can be a light spot or a shown object, for example.

A visual axis of the subject's eye can be varyingly displaced relative to an optical axis of the camera so that a blind spot of the light distribution of the pupil can be displaced in a direction differing to the optical axis of the camera through this visual movement. Due to a visual axis of the eye not perfectly aligning with or rather greatly varying relative to the optical axis of the camera, respectively, it becomes possible to displace the blind spot of the light distribution of the pupil, which occurs when the optical axis of the camera and the visual axis of the eye perfectly align, via the visual movement of the eye in a direction differing to the camera and consequently to determine a blind spot by capturing the pupil reflection or rather to also evaluate an ametropia of the blind spot, respectively.

Optionally, a gradient of a brightness distribution of a pupil reflex can be detected by means of a camera device and be determined by means of the control apparatus. The gradient of the brightness distribution can be used for determining the objective refraction of the eye in question even more exactly.

A pupil distance, a pupil diameter, a measuring distance, a tilt of the head and/or a viewing direction of the subject's eyes can be detected and measured by means of the camera device. If a subject's seeing distance to the monitor is principally known by positioning the monitor and the subject in a fixed position, the pupil distance can be evaluated by means of image processing an image recorded by the camera device or rather the camera of both of the subject's eyes. A relative distance of the pupil can, for example, be used for adjusting a pair of glasses or for conducting certain eye examinations. The pupil diameter can also be measured in said manner in dependence of a surrounding lighting. Vice versa, provided a pupil distance is known, a measuring distance or a subject's seeing distance can be evaluated relative to the monitor by means of the image processing of an image recorded by the camera device. A tilt of the subject's head relative to the monitor as well as a viewing direction or fixation of eye examination symbols, respectively, can also be detected.

Therefore, it is also particularly advantageous if a position, in particular an inclination, of the monitor relative to the subject's eyes can be measured by means of a position sensor of the display device. The position sensor can be a gyroscopic sensor by means of which a spatial position or rather a position of the monitor or rather the display surface can be determined. If, for example, the subject's eyes or head is recorded by means of the camera of the display device, an inclination of the monitor relative to the eyes can be easily evaluated when considering a known pupil distance. It can then also be shown on the monitor that the monitor is inclined relative to the eyes and an eye examination cannot be conducted, for example. Information on correctly adjusting the monitor relative to the subject's eyes can be shared on the monitor as well. The subject may then be able to adjust the monitor to the required position relative to their eyes in order to conduct an eye examination.

Continuous eye tracking of the subject's eyes can be conducted by means of the camera. Thus, a point of fixation of the eyes on a display surface of the monitor can be evaluated. This is possible when a viewing direction of the subject's eyes is detected. Thus, it can be examined within the scope of eye examinations, how dynamically the subject follows eye examination symbols shown in a monocular or binocular manner. Further embodiments of the method can be taken from the description of the device provided herein.

In the following, a preferred embodiment of the invention is further described in reference to the attached drawings.

### BRIEF DESCRIPTION OF THE DRAWINGS

In the drawings,

FIG. 1 shows a front view of an eye examining system;

FIG. 2 shows a schematic view of an illumination beam path of the eye examining system;

FIG. 3 shows a schematic view of an observation beam path of the eye examining system;

FIG. 4 shows a graphic view of a light distribution in a pupil relative to an ametropia.

FIG. 5 shows a block diagram of an eye examining system constructed in accordance with the present disclosure.

### DETAILED DESCRIPTION OF THE EXAMPLE EMBODIMENTS

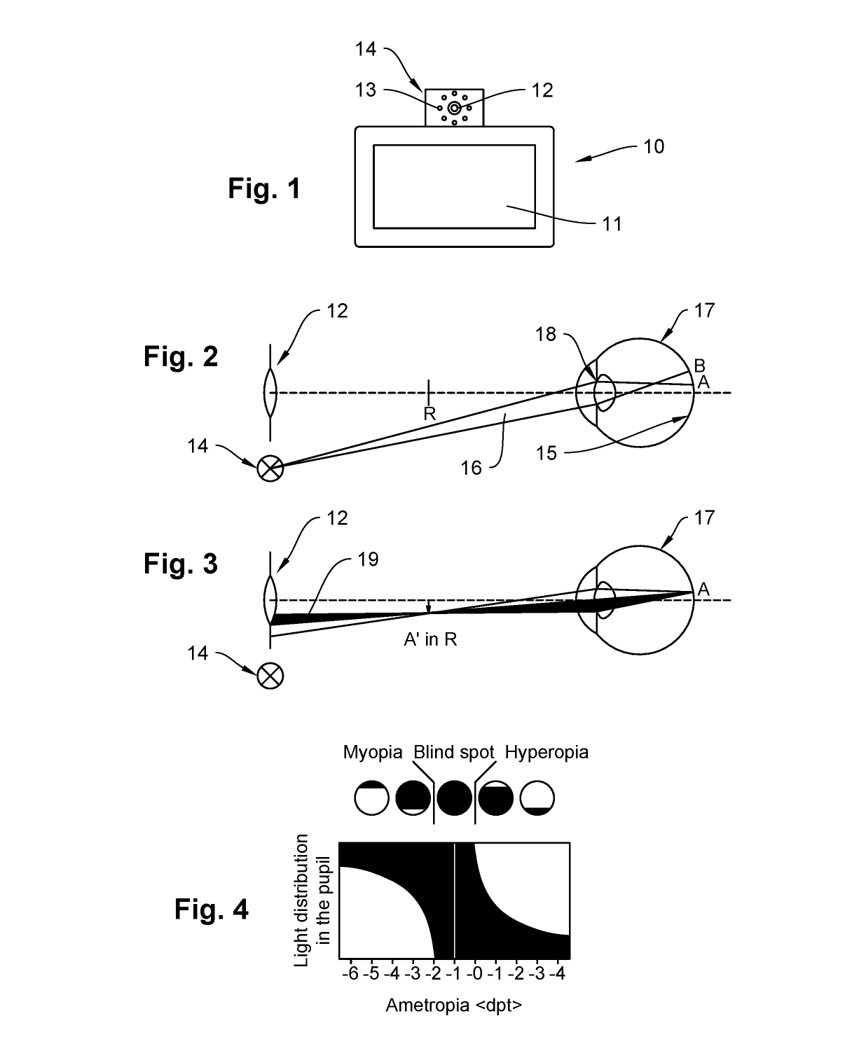

FIG. 1 shows a front view of an eye examining system 10 having a monitor 11, a camera 12 and a plurality of infrared light-emitting diodes 13, which are coaxially arranged relative to the camera 12. FIG. 5 shows a block diagram of an eye examining system 10 of the type described with reference to FIG. 1, and including a monitor 11, camera 12, an illumination or light source 14, and a corresponding control apparatus 23 and memory 24. In order to examine a subject's eyesight, it is regularly necessary to iteratively approach the ametropia of the subject's affected eye by showing eye examination symbols on a monitor and to subjectively determine a refraction in this manner, for example with a trial frame or a phoropter 25, shown by way of example in FIG. 5. It is also possible to first measure a separate objective refraction before measuring a refined subjective refraction. The objective refraction is measured comparatively exactly by means of an autorefractometer independently of the known subjective eye examining systems.

It is now intended to use the eye examining system 10 for first determining an objective refraction and for determining a subjective refraction directly following the objective measurement. The objective refraction is determined based on the so-called photo refraction principle, wherein a retina 15 or rather a fundus is illuminated by means of a light source 14, as shown in FIG. 2. Thus, a blurred image BA is obtained on the retina 15 in the illumination beam path 16, shown in an exemplary manner in FIG. 2, of a myopic eye 17. This image BA is recorded by the camera 12 through the pupil 18 of the eye 17 according to the observation beam path 19 shown in FIG. 3. This recorded light distribution of the pupil 18 or rather the pupil reflection are each different in dependence of the existing ametropia of the eye 17, as shown in FIG. 4. When examining the subject's eye 17, the eye 17 is therefore first illuminated with the light-emitting diodes 13 simultaneously or sequentially and a pupil reflection is recorded with the camera 12. From this pupil reflection, an ametropia or rather an objective refraction of the eye 17 by means of image processing can be evaluated by means of the control apparatus 23 , which serves to control the eye examining system 10, the monitor 11 and the camera 12. Directly after determining the objective refraction, which is inexact with respect to an autorefractometer in comparison, eye examination symbols can be shown to the subject on screen 11, said eye examination symbols being adjusted to the objectively measured refraction and thus allowing determining a subjective refraction without changing a measuring distance or the eye examining system 10.

For determining the objective refraction, a fixation stimulus can be shown on the monitor 11 or at a different position of the eye examining system 10 in such a manner that a visual axis 21 of the eye 17 does not perfectly align relatively to an optical axis 22 of the camera or greatly varies thereto, respectively. It then becomes possible to displace the blind spot of the light distribution of the pupil according to FIG. 4 in a direction varying to the camera 20 by the visual movement of the eye 17 and consequently detect the blind spot by capturing the pupil reflection or rather to also evaluate an ametropia of the blind spot, said blind spot occurring when the optical axis 22 of the camera 20 and the visual axis 21 of the eye 17 perfectly align.

1. An eye examining system for examining a subject's eyes comprising:
a display device displaying eye examination symbols to at least one of the subject's eyes, the display device comprising an illumination device having a light source configured to illuminate the at least one of the subject's eyes,
a camera device having a camera configured to record the at least one of the subject's eyes and a light distribution in the pupil of the at least one of the subject's eyes, and a control apparatus configured to determine an objective refraction of the eye from the light distribution in the pupil.

2. The eye examining system according to claim 1, wherein the display device, the camera and the light source are arranged in a shared housing of the eye examining system.

3. The eye examining system according to claim 1, wherein the display device is at least one of a stationary display device for testing farsightedness having a display surface sizes designed for conducting eye examinations at a seeing distance of 3 m to 10 m, and a mobile display device for testing nearsightedness having a display surface size designed for conducting eye examinations at a seeing distance of 10 cm to 3 m.

4. The eye examining system according to claim 1, wherein the display device comprises a backlit monitor.

5. The eye examining system according to claim 4, wherein the monitor forms the light source.

6. The eye examining system according to claim 1, wherein the light source is an infrared light source.

7. The eye examining system according to claim 1, wherein the light source is formed by a light emitting diode which is eccentrically arranged relative to an optical axis of a lens of the camera.

8. The eye examining system according to claim 7, wherein a plurality of light emitting diodes form the light source and coaxially surround the lens of the camera.

9. A method for examining a subject's eyes with an eye examining system comprising the steps of:
displaying eye examination symbols to at least one of the subject's eyes using a display device of the eye examining system,
illuminating the at least one of the subject's eyes with a light source in an illumination device in the display device,
recording the at least one of the subject's eyes with a camera of a camera device,
recording a light distribution in a pupil of the at least one of the subject's eye with the camera device of the display device, and
determining an objective refraction of the eye from the light distribution in the pupil with a control apparatus of the eye examining system.

10. The method according to claim 9, further comprising the step of controlling at least one of the light source and the camera with the control apparatus of the display device.

11. The method according to claim 9, further comprising the steps of determining the objective refraction of the at least one of the subject's eyes and directly after determining the objective refraction displaying eye examination symbols to the at least one of the subject's eyes and determining the subjective refraction with the eye examining system.

12. The method according to claim 9, further comprising the step of adjusting the eye examination symbols shown to the subject to the objective refraction.

13. The method according to claim 9, further comprising the step of choosing the eye examination symbols in dependence of the objective refraction.

14. The method according to claim 9, further comprising the step of determining the objective refraction based on the photo refraction principle.

15. The method according to, further comprising the steps of illuminating the eye is illuminated by light-emitting diodes simultaneously or sequentially and recording a corresponding pupil reflection with the camera.

16. The method according to claim 9, further comprising the step of presenting a fixation stimulus on at least one of a monitor of the display or at a different position of the eye examining system for determining the objective refraction.

17. The method according to claim 9, further comprising the step of varyingly displacing a visual axis of the subject's eye relative to an optical axis of the camera in such a manner that a blind spot of the light distribution in the pupil of the at least one of the subject's eyes is displaced by this visual movement of the eye in a direction varying relative to the optical axis of the camera.

18. The method according to claim 9, further comprising the step of detecting a gradient of a brightness distribution of a pupil reflex with the camera device and determining the gradient of the brightness distribution of the pupil reflex with the control apparatus.

19. The eye examining system according to claim 1, wherein the display device comprises at least one of a television, a monitor, and a tablet computer.
